(19) United States
(12) Patent Application Publication
(10) Pub. No.: US 2021/0268212 A1
(43) Pub. Date: Sep. 2, 2021

(54) BREATH MEASUREMENT DEVICE

(71) Applicant: Christopher V. CIANCONE, Layton, UT (US)

(72) Inventor: Christopher V. CIANCONE, Layton, UT (US)

(21) Appl. No.: 17/259,140

(22) PCT Filed: Jul. 10, 2019
(86) PCT No.: PCT/US2019/041153

§ 371 Date: Jan. 8, 2021

Related U.S. Application Data
(60) Provisional application No. 62/696,243, filed on Jul. 10, 2018.

(51) Int. Cl. A61M 15/00; G06N 20/00; G16H 10/60; G16H 40/40; G16H 40/67; G16H 20/10; G16H 50/70

(52) U.S. Cl. CPC A61M 15/0001; G16H 50/20; A61M 15/0021; A61M 15/0083; A61M 15/009; G06N 20/00; G16H 10/60; G16H 40/40; G16H 40/67; G16H 20/10; G16H 50/70; A61M 2205/3334; A61M 2205/70; A61M 2205/52; A61M 2205/581; A61M 2205/3327; A61M 2205/582; A61M 2205/583; A61M 15/0086

(57) ABSTRACT

An inhalation interface may monitor and analyze flow rate in an inhalation device. The inhalation interface may be calibrated for a user. The inhalation interface may send usage data to a user for review. In some embodiments the inhalation interface may send usage data to a physician and provide active feedback to a patient. The active feedback may indicate when the flow rate during an inhalation is within a target threshold.

### TECHNICAL FIELD

This disclosure relates to an inhalation device with data tracking and feedback mechanisms.

### BRIEF DESCRIPTION OF THE DRAWINGS

FIG. 1 illustrates a block diagram of an analysis device configured to measure inhalation factors, transmit the measurements, and provide feedback to a patient during treatment.

FIG. 2 illustrates a flowchart of a method for processing a pressure signal and providing feedback based on the pressure signal.

FIG. 3 illustrates a flowchart of a method for an analysis device to capture and transmit usage data.

### DETAILED DESCRIPTION OF PREFERRED EMBODIMENTS

Respiratory therapy often relies on pressurized Metered Dose Inhalers (pMDI) or Soft Mist Inhalers (SMIs) to deliver medicine into the lungs for ailment treatment. Proper inhalation rate and timing, among other factors, assist with delivery of MDI and SMI respiratory medicines. Typically, patients are trained by a clinician (respiratory therapist, pharmacist, physician, etc.) on proper breath technique to ensure maximum drug delivery into the lungs. Costly training and evaluation respiratory therapy sessions are limited to clinical settings and the training effect wears off as the patient develops sub-optimal breathing habits outside the clinic. Some inhaler spacer chambers include a noise-maker reed valve calibrated to make a noise at the proper flow rate, but these have been shown to be poorly calibrated and prone to errors.

Real time inhalation technique feedback during routine medicine application will increase medicine delivery rates and increase the quality of respiratory ailment treatment. Some embodiments herein include an inhalation interface comprising an analysis device that measures factors associated with an inhalation, provides real-time feedback to the patient, and transmits/stores data for trend analysis. The analysis device may be integrated into and/or attached on to an inhaler spacer chamber, intubation spacer, MDI, or other respiratory therapeutic device. Some examples of respiratory therapeutic devices include MDI inhalers, portable and clinical nebulizers, typical MDI spacers, and the LIYEN™ inhaler spacer system. The analysis device may be integrated in embodiments described in US Publication Number 2016/0022933, entitled Inhaler Spacer and Storage Apparatus, which is hereby incorporated by reference in its entirety.

Inhalation devices are used in a number of applications. For example, recreational inhalable devices include cannabidiol (CBD) devices, vape pens, and e-cigarettes. Inhalation devices also include medical inhalable devices to deliver a drug. For example, medical inhalable devices include inhalers and nebulizers. Medical inhalable devices may be used to deliver drugs to treat a number of conditions including asthma, cancer, and diabetes. Inhalation devices may also be used to measure a user's inhalation rate rather than delivery of a drug. For example, inhalation devices may be used as a tracking device. For example, a tracking device may be used to record an athletes lung inhalation rate during an exercise, or oxygen consumed by a scuba diver. While this application describes many example embodiments in relation to an inhaler or inhaler spacer and patient, the systems, devices, and methods may also be applied to recreational inhalable devices, medical inhalable devices, and tracking inhalation devices and their users.

The analysis device may comprise a sensor package, battery subsystem, user input interface, and an output interface. The analysis device is to measure breath statistics with a sensor, process the data (analyze, store, compare, and transmit), provide feedback to the user, and transmit/store data for trend analysis by user or care provider. This may be accomplished through a combination of core components, integration options, back-end data analysis algorithm, and feedback indicia options. The analysis device may be configured as an Internet of Things (IoT) device and serve as a data interface for another device or be controlled by another device.

Some of the infrastructure that can be used with embodiments of the analysis device and/or for trend analysis is already available, such as: general-purpose computers, computer programming tools and techniques, digital storage media, and communications networks. A computer may include a processor, such as a microprocessor, microcontroller, logic circuitry, or the like. The processor may include a special-purpose processing device, such as an ASIC, PAL, PLA, PLD, FPGA, or other customized or programmable device. The computer may also include a computer-readable storage device, such as non-volatile memory, static RAM, dynamic RAM, ROM, CD-ROM, disk, tape, magnetic memory, optical memory, flash memory, or another computer-readable storage medium.

Suitable networks for configuration, the input interface, and/or the output interface, as described herein, include any of a wide variety of network infrastructures. Specifically, a network may incorporate landlines, wireless communication, optical connections, various modulators, demodulators, small form-factor pluggable (SFP) transceivers, routers, hubs, switches, and/or other networking equipment. The network may include communications or networking software, such as software available from Novell, Microsoft, Artisoft, and other vendors, and may operate using UDP, TCP/IP, SPX, IPX, SONET, and other protocols over twisted pair, coaxial, or optical fiber cables; telephone lines; satellites; microwave relays; modulated AC power lines; physical media transfer; wireless radio links; and/or other data transmission “wires.” The network may encompass smaller networks and/or be connectable to other networks through a gateway or similar mechanism.

Aspects of certain embodiments may be implemented as software modules or components. As used herein, a software module or component may include any type of computer instruction or computer executable code located within or on a computer-readable storage medium. A software module may, for instance, comprise one or more physical or logical blocks of computer instructions, which may be organized as a routine, program, object, component, data structure, etc., which perform one or more tasks or implement particular abstract data types. A particular software module may comprise disparate instructions stored in different locations of a computer-readable storage medium, which together implement the described functionality of the module. Indeed, a module may comprise a single instruction or many instructions, and may be distributed over several different code segments, among different programs, and across several computer-readable storage media.

Some embodiments may be practiced in a distributed computing environment where tasks are performed by a remote processing device linked through a communications network. In a distributed computing environment, software modules may be located in local and/or remote computer-readable storage media. In addition, data being tied or rendered together in a database record may be resident in the same computer-readable storage medium, or across several computer-readable storage media, and may be linked together in fields of a record in a database across a network. According to one embodiment, a database management system (DBMS) allows users to interact with one or more databases and provides access to the data contained in the databases.

In the following description, various aspects of the illustrative implementations will be described using terms commonly employed by those skilled in the art to convey the substance of their work to others skilled in the art. However, it will be apparent to those skilled in the art that the disclosure may be practiced with only some of the described aspects. For purposes of explanation, specific configurations are set forth in order to provide a thorough understanding of the illustrative implementations. However, it will be apparent to one skilled in the art that the disclosure may be practiced without the specific details. In other instances, well-known features are omitted or simplified in order not to obscure the illustrative implementations.

Various operations will be described as multiple discrete operations, in turn, in a manner that is most helpful in understanding the disclosure; however, the order of description should not be construed to imply that these operations are necessarily order dependent. In particular, these operations need not be performed in the order of presentation.

Additional details and examples are provided with reference to the figures below. The embodiments of the disclosure can be understood by reference to the drawings, wherein like parts are designated by like numerals throughout. The components of the disclosed embodiments, as generally described and illustrated in the figures herein, could be arranged and designed in a wide variety of different configurations. Thus, the following detailed description of the embodiments of the systems and methods of the disclosure is not intended to limit the scope of the disclosure, as claimed, but is merely representative of possible embodiments.

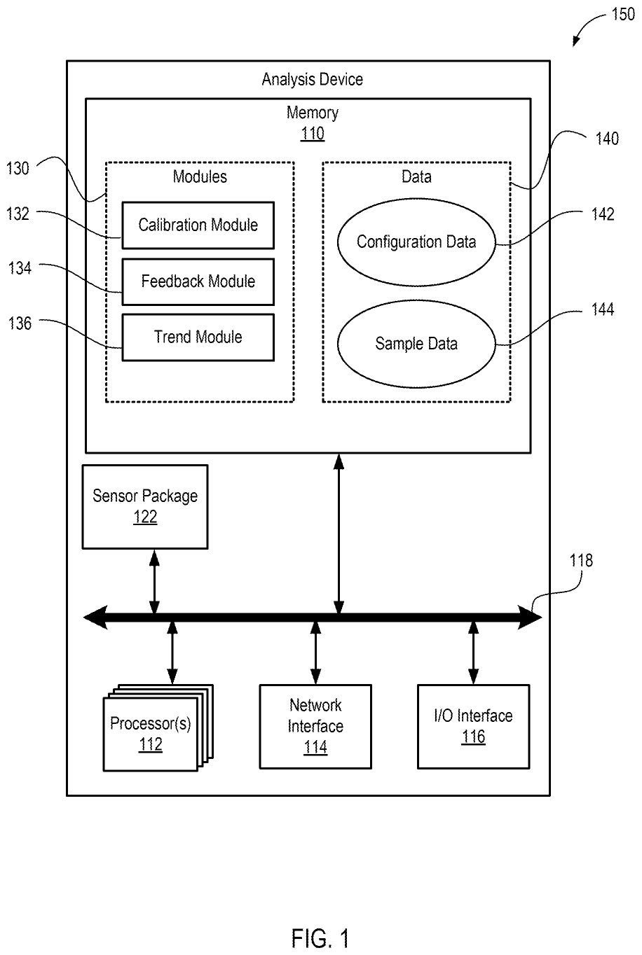

FIG. 1 illustrates a block diagram of an analysis device 150 configured to measure inhalation factors, transmit the measurements, and provide feedback to a user. The analysis device 150 may be a part of an inhalation interface configured to interface with an inhalation device. The analysis device 150 may include a printed circuit board (PCB) with a microcontroller, clock generator, voltage regulator, and associated components for microcontroller operation. A fully integrated CPU may also be used. As shown, in some embodiments, the analysis device may comprise include an electronic memory 110, one or more processors 112, a network interface 114, an I/O interface 116, and a sensor package 122.

The analysis device 150 may be incorporated into an inhalation interface. For example, the analysis device 150 may be integrated into various devices at a number of positions. For instance, the analysis device 150 may be within a mouthpiece or a spacer of an MDI device. In some embodiments, the analysis device 150 may be integrated into an adapter that can be added onto a device. The analysis device 150 may be used with recreational inhalable devices, medical inhalable devices, or inhalation tracking devices. The analysis device 150 may be incorporated into a portion of these inhalation devices, or may be integrated into an adaption device that interfaces with an inhalation devise.

In some embodiments, the inhalation device may be a portable device. In some embodiments, the inhalation interface may be configured to attach or be integrated into the portable device.

The sensor package 122 includes a pressure sensor attached to a PCB or remotely mounted off the PCB. The pressure sensor is integrated in such a way as to have direct or indirect contact with internal gas volume of an inhalation device such as a spacer. In one embodiment, the pressure sensor senses flow pressure corresponding to the air flow speed according to Bernoulli's principle. The pressure sensor directly measures absolute pressure (P) in the internal volume. As inhalation occurs, P drops proportionally to the velocity (u) according to

$$u = \sqrt{\frac{2 ( {P}_{1} - {P}_{2} )}{ρ}}$$

where P1 is the initial ambient pressure, P2 is the pressure during inhalation, and ρ is the density of the air. Air density is approximated using pressure and temperature according to the ideal gas law

$$ρ = \frac{{P}_{1}}{R T}$$

where P1 is the initial ambient pressure, R is the specific gas constant, and T is the air temperature. With the velocity calculated, the volumetric flow rate (Q) is calculated by Q=uA where u is the measured velocity and A is the cross-sectional area. Temperature may be based on data from an external device, location information, or a temperature sensor included in the sensor package 122. For example, the analysis device 150 may receive a temperature from a user's personal electronic device.

In some embodiments, the sensor package 122 includes one or more sensors in addition to the pressure sensor. For example, the sensor package 122 may include a temperature sensor, an atmospheric pressure sensor, pollen sensor, and/or location sensor (e.g., global positioning system transceiver). The environmental sensors may measure one or more of air particulates, location, pollen levels, air quality, atmospheric pressure, atmospheric conditions, temperature, time, and date

In some embodiments, the sensor package 122 includes a sensor to detect when a pressurized MDI canister is depressed. In some embodiments, the sensor package 122 may include a sensor to detect the type of drug being administered. In some embodiments, the sensor package 122 may include a motion sensor that may be used to determine activity level of the user.

The electronic memory 110 may include static RAM, dynamic RAM, flash memory, one or more flip-flops, or other electronic storage medium. The electronic memory 110 may include a plurality of modules 130 and data 140. The modules 130 may run multiple operations serially, concurrently or in parallel by or on the one or more processors 112.

In some embodiments, portions of the disclosed modules, components, and/or facilities are embodied as executable instructions embodied in hardware or in firmware, or stored on a non-transitory, machine-readable storage medium. The instructions may comprise computer program code that, when executed by a processor and/or computing device, cause a computing system to implement certain processing steps, procedures, and/or operations, as disclosed herein. The modules, components, and/or facilities disclosed herein may be implemented and/or embodied as a driver, a library, an interface, an API, FPGA configuration data, firmware (e.g., stored on an EEPROM), and/or the like. In some embodiments, portions of the modules, components, and/or facilities disclosed herein are embodied as machine components, such as general and/or application-specific devices, including, but not limited to: circuits, integrated circuits, processing components, interface components, hardware controller(s), storage controller(s), programmable hardware, FPGAs, ASICs, and/or the like.

The modules 130 may include, a calibration module 132, a feedback module 134, and a trend module 136. The calibration module 132 may receive input from the sensor package 122, the network interface 114, and/or the I/O interface 116. The input may include a user profile, environmental conditions, an inhalable profile, a device profile, historic activity level, current activity level, and/or prescription information.

A user profile contains data representing the user. For example, the user profile for a medical inhalation device may include patient information including height, weight, age, prescription data, drug information, and/or dosage information. The user profile may include other health information such as resting heart rate, lung capacity, and/or activity level. The user profile may be generated by receiving information from a user or physician. For example, a user may enter a user profile into an application on an electronic device such as a smart phone, computer, personal electronic device, or other digital device that is then sent to the analysis device 150.

An inhalable profile describes characteristics of an inhalant. Inhalable profiles may include a dry powder profile, a soft mist profile, a nicotine profile, a CBD profile an alcohol based drug profile. Each of these inhalants may behave differently during delivery. The profiles provide information about the delivery behavior. In some embodiments, the inhalable profile may include the inhalable chemical content, propellant content/type, inhalant particulate size and characteristic, drug type, and drug composition, and inhalable composition.

A device profile is a representation that characterizes the inhalation device. For example, the device profile may include whether or not the inhalation device includes a spacer, the size of the spacer, the size of the mouthpiece, and/or the type of inhalant typically used. For example, if the inhalation device is a vape pen, the device profile may include the size and shape of the device, and that E-juice is the typical inhalant. In some embodiments, the typical inhalant of the device profile may be used to determine which inhalable profile the analysis device 150 should use.

The calibration module 132 may, by the one or more processors, perform operations to calibrate the analysis device 150 for the user and/or the environment based on the input. The calibration may be based on one or more of measurements from the sensor package, the device profile, the inhalable profile, and the user profile. The calibration may include inhalation rate, dosage amount, and/or treatment schedule. In some embodiments, to calibrate the analysis device 350 the sensor package 122 may include a sensor to detect the type of drug in an MDI canister. In some embodiments, the drug information may be input by user. In some embodiments, the drug information may be detected using a NFC tag, a barcode, or other identifier. A user may input information for calibration via the network interface 314 with another device such as a smartphone or other personal electronic device.

In some embodiments, the device profile, the inhalable profile, and the user profile may include a data architecture that correlates environmental data to data collected during usage in that environment. For example, the analysis device may correlate an altitude of a runner and measurements from a pressure sensor in the runner's mouthpiece. In some embodiments, the calibration module 132 may calibrate the inhalation device by identifying previous environmental conditions recorded in the user profile, the device profile, or the inhalable profile that are similar to the current measurements from the environmental sensors, and identify a flow rate in the user profile that is correlated to the previous environmental conditions. If there is not an exact match between the previous environmental conditions and the current environmental conditions, the analysis device may extrapolate a calibration information from multiple previous records. The flow rate may be used as a data point for the calibration module 132 to calibrate the inhalation device.

The feedback module 134 may, by the one or more processors, provide an output to aid a user based on the calibration. In some embodiments, the feedback module 134 may send data to a personal electronic device for the user to review his or her progress. For example, an athlete may desire a greater flow rate during an exercise.

In a medical inhalation device, the feedback module 134 may provide an output to aid a patient in treatment based on the calibration for the user and conditions. For example, the feedback module may provide a delivery dose reminder. When a user is due for a dose the feedback module 134 may cause the I/O interface 116 to provide visual, audio, and/or haptic feedback. For instance, the I/O interface 116 may cause an LED light to illuminate, a speaker to make a noise, or a motor to vibrate. In some embodiments, the feedback module 134 may communicate with the network interface 114 to cause a personal device associated with the user to alert the user. In some embodiments, the inhalation interface or inhalation device may be at least semi-translucent, and an led inside the inhalation interface or inhalation device may illuminate when a user is due for a dose causing the inhalation interface or inhalation device to glow.

The feedback module 134 may also determine additional diagnostic data to generate a user inhalation profile. The inhalation profile includes data obtained during use of an inhalation device. The use of the inhaltion device may include inhaltion and exhalation by the user. For example, the user inhalation profile may be used to determine lung volume, pulse, heart rate, and electrolyte levels. In some embodiments, the feedback module 134 may correlate the flow rate with additional diagnostic data from other devices such as a heart rate sensors. In some embodiments the feedback module 134 may be used to determine various characteristics of a user's breath, which can be used to diagnose certain conditions.

The feedback module 134 may also provide a user with active feedback during use. For example, a feedback module 134 used in an inhaler may be used for feedback during treatment. For instance, the feedback module 134 may compare the inhalation rate during treatment to the calibrated inhalation rate and provide corrective feedback to the user if the inhalation rate is different than the calibrated inhalation rate. For example, the feedback module may cause the I/O interface 116 to provide visual, audio, and/or haptic feedback. For instance, the I/O interface 116 may cause an LED light to illuminate, a speaker to make a noise, or a motor to vibrate. In some embodiments, the feedback module 134 may communicate with the network interface 114 to cause a personal device associated with the user to provide active feedback to the user.

The trend module 136 may record data associated with the inhalation device usage such as, but not limited to data records, such as time, date, time and/or date of treatment, treatment duration, airflow resistance settings, flow rate, flow volume, number of dosages used and unused, dosage amounts, medicament information, such as name and serial number, breathing pattern information, user's progress, device program information, such as device temperature settings, frequency settings, airflow settings, timing settings, aerosolization settings for a particular type of medicament, and other user settings. Additionally in some embodiments, the trend module 136 may track activity level of a user and environmental conditions measured by the sensor package 122. In some embodiments, the trend module 136 may store the data in memory 110, transmit the data 140 via the network interface 114, or analyze the data for trends.

In some embodiments, the trend module 136 may be used to update a treatment plan for a patient. For example, the trend module 136 may send the usage data to a physician who may review the data and update the dosage schedule or dosage amount. In some embodiments, the trend module 136 may use machine learning to determine an optimum treatment plan for the patient. The optimum treatment plan may be sent to a physician for approval. The updated treatment plan may be recorded in the user profile and used during a future calibration.

In some embodiments, the feedback module 134 or the trend module 136 may aggregate the data from the sensor package 122 and correlate the measurements from the environmental sensors and the measurement of flow rate to generate or update a user profile. The user profile may become a data structure that provides a representation of a user during various measured environmental conditions. The calibration module 132 may re-calibrate the analysis device 150 based on the updated user profile to provide a more accurate calibration. Similarly, the feedback module 134 or the trend module 136 may update the inhalable profile or device profile for future calibrations.

In some embodiments the sensor package 122 may include additional breath analysis sensors to determine various characteristics of a user's breath. For example, the additional sensors may extrapolate biological data of the user from samples captured during use of the inhalation device to generate a biological profile of a user. The biological profile of the user may indicate a biological status of the user. For instance, the sensors may be used to perform breath analysis to diagnose diseases, determine hydration levels, determine electrolyte levels, determine body temperature levels, determine oxygen saturation levels, determine glucose levels, determine potassium levels, and determine other biological chemistry and conditions. In some embodiments, a breath analysis sensor may include a flow rate sensor such as a pressure sensor. The biological user profile may be used to provide feedback to a user. For example, biological user profile may be used to generate dosage reminders, usage recommendations, active usage feedback, training schedule, etc. In some embodiments a usage recommendation may include a usage schedule and amount. In some embodiments, the usage recommendation may be used to determine usage reminders.

The data 140 stored on the electronic memory 110 may include the data 140 generated by the processing circuitry 150, such as by the modules 130 or other modules. The data 140 stored may be organized as one or more memory registers/addresses, files, and/or databases. The data 140 may include configuration data 142 and sample data 144. The configuration data 142 may include user settings, such as alarm settings, password protection, and prescription information. The sample data 144 may be information associated with MDI usage such as, but not limited to data records, such as time, date, time and/or date of treatment, treatment duration, airflow resistance settings, flow rate, flow volume, number of dosages used and unused, dosage amounts, medicament information, such as name and serial number, breathing pattern information, user's progress, device program information, such as device temperature settings, frequency settings, airflow settings, timing settings, aerosolization settings for a particular type of medicament, and other user settings.

The one or more processors 112 may include general purpose processors and/or special purpose processors. In one embodiment, the one or more processors 112 include a LoRa® chip and/or a Bluetooth® chip to provide special purpose transmit (Tx) and/or receive (Rx) functionality for communicating with other computing devices. These special purpose Tx/Rx chips may supplement and/or be included in the network interface 114. The Tx/Rx chips may be used by the proximity sensor 122 to measure a signal strength between a PED and the tracking device.

The network interface 114 may facilitate communication with other computing devices and/or networks, such as the Internet and/or other computing and/or communications networks. The network interface 114 may be equipped with conventional network connectivity. The network interface 114 may be a wireless network interface, equipped with conventional wireless network connectivity technologies. The network interface 114 may facilitate communication with a trend analysis database that compares trends of the user overtime. For example, a physician may receive monitor this data to adjust treatment. A coach may receive this data and implement changes to an athletes training schedule. In some embodiments, the analysis device 150 is configured as an IOT device and the network interface 114 may be configured to communicate with cellular networks (e.g., 4G and/or 5G). The network interface 114 on an IOT analysis device may enable the analysis device to transmit data to an application on a personal electronic device to provide a data interface. The network interface 114 can also allow the application to control the device and serve as a function interface.

The I/O interface 116 may facilitate interfacing with the user. The I/O interface 116 may be any suitable human machine interface. For example, the I/O interface 116 may comprise a display, lights, vibration motor, or speaker to allow the analysis device 150 to output information to the user. For example the I/O interface 116 may include a first light to instruct the user to increase inhalation rate and a second light to instruct the user to decrease inhalation rate. Auditory or haptic feedback may also be used.

A system bus 118 may facilitate communication and/or interaction between the other components of the processing circuitry 150, including the electronic memory 110, the one or more processors 112, the network interface 114, the I/O interface 116, and the sensor package 122.

As can be appreciated, in other embodiments, the processing circuitry 150 may be simpler than shown or described. For example, certain designs may forgo one or more components, such as memory, multiple processors, multiple interfaces, and the like, and instead execute instructions closer to or on bare metal (e.g., without intervening operating system or other software layer, executing instructions directly on logic hardware).

The analysis device 150 may be powered by a battery. In some embodiments, the battery is positioned on the back of the PCB or in another accessible location if integrated into an inhalable device such as a spacer. The battery may be user replaceable via an access door or battery slot drawer. If mounted off the PCB, wires integrated into a spacer or adhered to the spacer body connect the battery to the analysis device 350. The battery may also be integrated and not user serviceable and rechargeable via USB, induction charging, kinetic charging system, or solar powered among other options.

The analysis device may be integrated in a mouthpiece, a recreational inhalable device, a medical inhalable device, a inhalation tracking device, a spacer, or an MDI. The analysis device 150 may be incorporated into a portion of these inhalation devices, or may be integrated into an adaption device that interfaces with an inhalation device. For example, the analysis device may be integrated into an EMT intubation mask, a clinical respirator, an oxygen mask, a tracheostomy breathing assembly, a divers mouthpiece, a divers breathing apparatus, an emergency personnel's breathing apparatus, an athlete's mouthpiece, a resistance training masks, or a climber's breathing apparatus. The analysis device may track oxygen flow rate based on the sensor measurements.

In some embodiments, the analysis device 150 may be integrated in a jacket for an MDI. The jacket may be a lever assisted device configured to provide assistance in depressing the MDI. For example, a lever may depress the MDI canister when a user squeezes the jacket.

The analysis device 150 may be integrated into a sub-compartment of an inhalable device. For example, the analysis device 150 may be integrated into a mouthpiece for scuba diving. The analysis device 150 may be integrated into a sub-compartment in a spacer chamber. This allows for flexible manufacturing and/or aftermarket feature enhancement of an existing spacer or integration into an evolving shape/style of an existing model spacer. In some embodiments, a small pressure port connects the sensor package air volume to the chamber air volume. The pressure port may be a straight pass through or contain a serpentine channel to prevent aerosol contamination of the pressure sensor.

In some embodiments, the analysis device 150 may be integrated into a nebulizer mouthpiece or nebulizer type device. In some embodiments, the analysis device is manufactured into the nebulizer mouthpiece or nebulizer type device. In other embodiments, the analysis device may be an accessory that may be selectively inserted into a nebulizer mouthpiece or nebulizer type device.

User inputs are positioned either integrated into the analysis device housing or removed from the sensor package PCB. The user inputs may be mechanical buttons, capacitance switch, or other input. In some embodiments, the user inputs may be sent from an external device and received via communication circuitry. For example, a smart phone may connect with the analysis device via Bluetooth or Wi-Fi to receive user inputs. The inputs allow a user to power on the device and change measurement modes among other settings. Output of the breath measurement information is accomplished by one or multiple indicia such as a LCD screen, eInk display, haptic device, Bluetooth link to an external device (e.g., smartphone, tablet, computer), or an auditory signal. The indicia may be positioned on the PCB package or remotely and may also be integrated into the user input buttons.

In another example of the analysis device 150, the pressure sensor is mounted into the mouthpiece of an inhalable device. For example, the pressure sensor may be mounted into the mouthpiece of an inhaler spacer or on the top of an inhaler body to directly measure flow into the mouth. The microcontroller PCB is mounted away from the pressure sensor to maintain a clean environment aesthetic around the mouthpiece. In this example the sensor package and microcontroller may be fully integrated into an inhaler spacer. When integrated into the mouthpiece, the mouthpiece may or may not be flexible and retractable into the spacer chamber body.

In another example of the analysis device 150, the unit is contained in a self-contained enclosure. This enclosure may mount onto an inhalable device. For example, this enclosure may mount onto an MDI inhaler body mouthpiece, spacer chamber mouthpiece, or on the top of an inhaler body. The unit includes a flow pass through where the medicine aerosol passes through the mouthpiece extension unit containing the pressure sensor package. In the case of the inhaler top cap, the unit will measure incoming air passing by the MDI canister. This unit can be adapted to a variety of existing spacer chamber, MDI/SMI medicine device, or nebulizer mouthpiece. As a standalone device, the unit may include all sensor, input, output, and battery components within one physical enclosure.

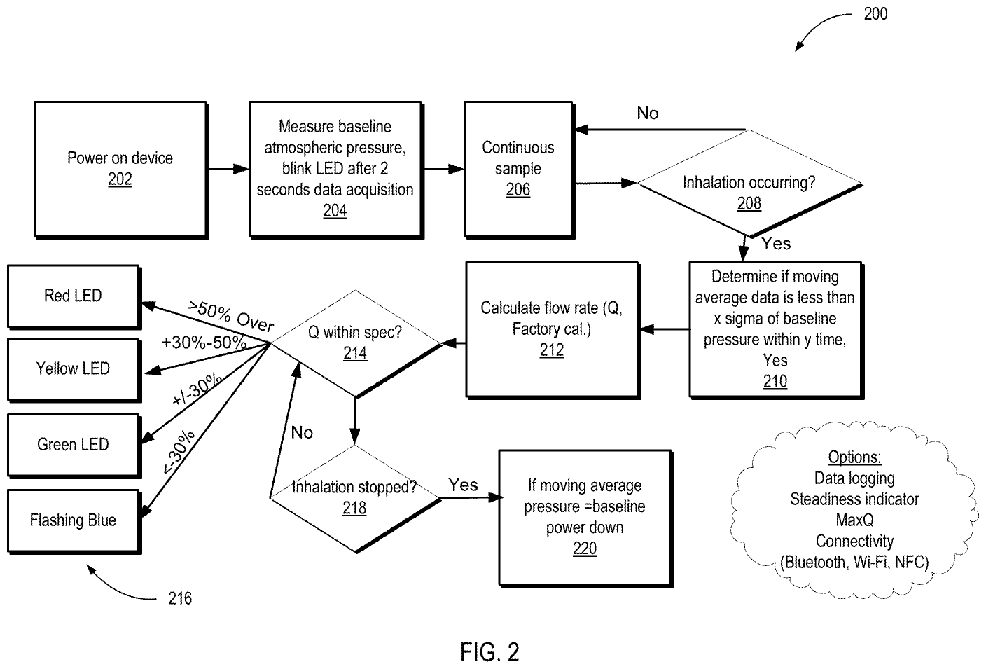

FIG. 2 illustrates a flowchart of a method 200 for processing a pressure signal and providing feedback based on the pressure signal. In certain embodiments, the processing of the pressure signal into volumetric flow rate (Q) indicators to the user is accomplished with an algorithm performed on the integrated microcontroller or processed remotely on a server. The user will activate the device to turn on 202 the measurement system and an indicator will indicate 204 that the sensor is ready. Activation may occur by any of several means including the user pressing a button, touch panel, opening a device, shaking the device to actuate an accelerometer in the microcontroller, or an application on the user's personal electronic device. For example, activation may occur when an inhaler spacer and storage apparatus is opened. During this short startup time, the sensor will be establishing an average atmospheric pressure as a reference value. For example, the sensor may take 206 a continuous sample.

The user will then actuate the inhaler and proceed to inhale 208 through the spacer mouthpiece. The device may determine 210 that inhalation has occurred if the moving average data is less than the baseline pressure within a certain time. The device may calculate 212 a flow rate and determine 214 if the flow rate is within a specified range. During the inhalation, the indicator will indicate 216 the volumetric flow rate, peak flow, duration, or any other inhalation measurement metric. For example, a LED will flash if the speed is too low, and/or change color based on the inhalation parameters. The indicator may have green, yellow and red lights indicating, acceptable, marginal, and over speed limits of the inhalation flow rate. The device may determine 218 if inhalation has stopped based on a comparison between sample data and baseline pressure. After inhalation, the device will shut down 220 automatically to preserve battery.

In the background, the microcontroller package is performing a series of calculations, calibrations, and decisions. During initial startup, the mean and standard deviation of the atmospheric pressure and temperature will be saved. Once sufficient samples have been saved, the indicator indicates ready. In the ready mode, the sensor will continually sample (˜20-200 samples per second or other appropriate programmed rate) and establish a short moving average to smooth any spurious data. An inhalation will be detected by comparing a short moving average with the mean atmospheric pressure, if the moving average is less than X standard deviations of background for a duration of Y time, then an inhalation event has started. During the inhalation event, the pressure signal will be converted into a volumetric flow rate (Q) based on factory calibrations or user settings. These Q rates will then be compared with established optimal flow rates and the feedback will be indicated to the user. For example, depending on the measured and smoothed Q data, an LED will change from flashing blue, green, yellow, and red if Q is low, good, high, or very high respectively. Once the pressure signal returns to the baseline atmospheric pressure for an established period of time, the microcontroller measures that the inhalation event has ended and the device will shut down to preserve battery. All algorithm details may be changed due to further device development and/or established industry and clinical best practice improvements.

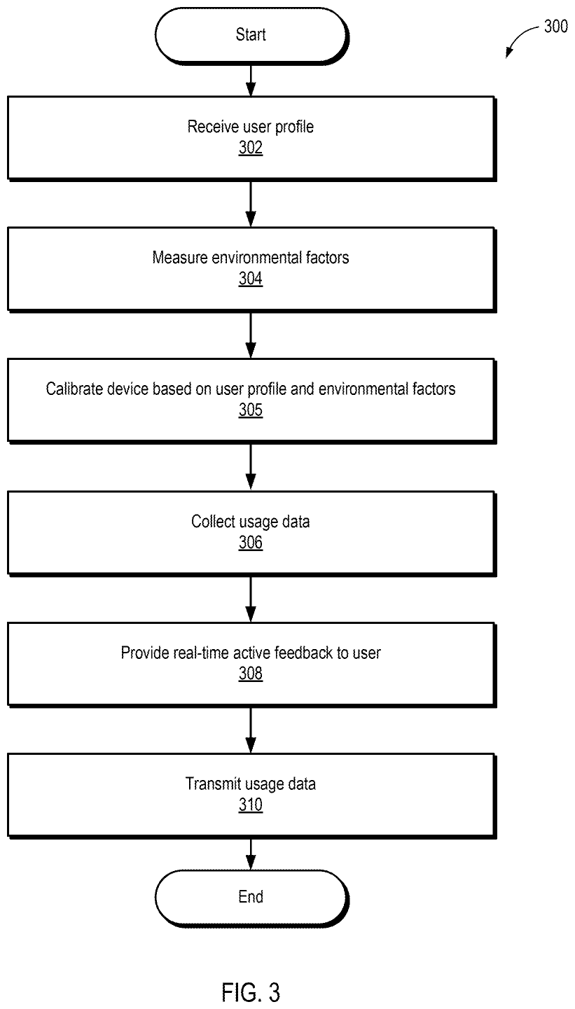

FIG. 3 illustrates a flowchart of a method 300 for an analysis device to capture and transmit usage data. Each embodiment described with reference to the previous figures may be configured with wired and/or wireless connectivity. The method 300 may include receiving 302 a user profile. The user profile may include user height and weight, prescription data, drug information, and dosage information. The method 300 may also measure 304 environmental factors such as location, pollen levels, air quality, atmospheric pressure, temperature, time, and date. The method 300 may also include calibrating 305 the device based on the user profile and the environmental factors. In some embodiments, the calibration may also be based on a device profile and an inhalable profile. During inhalation, the method may collect usage data 306 and provide real-time active feedback 308 to the user. For example provide feedback to the user based on flow rate.

In some embodiments, the usage data may be used as feedback to update the user profile, inhalable profile, or device profile, such that the analysis device may be more accurately calibrated during future uses in similar environmental conditions. The method may comprise updating these profiles by recording collected usage data including the measurement from the flow rate sensor and recording environmental factors. The method may further include determining the accuracy of the calibration by examining actual measurements to expected measurements. The results of the comparison may be used to update a user profile, a device profile, and/or an inhalable profile. For example, the method may compare an amount of drug actually delivered compared to an expected drug delivery for a given flow rate and update a device profile based on this comparison. The method may further include correlating the measurements and environmental factors into a data structure that allows access for re-calibration.

Usage data 306 includes data collected during an inhalation event (inhalation event data) including environmental factors. In some embodiments, the usage data 306 includes data concerning the timing or pattern of the inhalation event in relation to previous inhalation events.

Inhalation event data (e.g., flow rate time series, averaged values, timings, time, date, time and/or date of treatment, treatment duration, airflow resistance settings, flow rate, flow volume, number of dosages used and unused, dosage amounts, medicament information, such as name and serial number, breathing pattern information, user's progress, device program information, such as device temperature settings, frequency settings, airflow settings, timing settings, aerosolization settings for a particular type of medicament, and other user settings) will be transmitted 310 via USB, Bluetooth, WiFi, NFC, or another protocol to a user electronic device (phone, tablet, PC, handheld computing device, etc). The particular communication protocol hardware is contained on the microcontroller sensor package PCB. The data may also be stored locally on non-volatile memory. The data will be available to establish treatment trends and therapy feedback to the user both in clinical and other user environments. From the user electronic device, the data may be transmitted securely over the internet to a supervising physician (respiratory therapist, nurse, doctor, etc) third party, clinic, facility, etc.

### Examples

The following examples pertain to further embodiments.

Example 1: an device to collect usage data of an inhaler, the device comprising: a pressure sensor to take measurements including measurements of flow rate in a spacer; a network interface; and a processing unit to: receive patient information; calibrate the device based on the patient information; provide active feedback to patient during an inhalation, wherein the active feedback is adjusted based on the calibration; collect usage data; and transmit, via the network interface, the usage data.

Example 2: The device of claim 1, further comprising additional sensors, and wherein the processing unit is further to measure environmental factors via the additional sensors and calibrate the device based on the environmental factors.

Example 3: The device of claim 2, wherein the environmental factors include location, pollen levels, air quality, atmospheric pressure, temperature, time, and date.

Example 4: The device of claim 1, wherein the patient information includes at least one of height, weight, prescription data, drug information, and dosage information.

Example 5: The device of claim 1, further comprising a vibration motor, wherein the active feedback comprises vibration.

Example 6: The device of claim 1, further comprising a speaker, wherein the active feedback comprises sound.

Example 7: The device of claim 1, further comprising one or more LEDs, wherein the active feedback comprises changing an output of the one or more LEDs.

Example 8: The device of claim 1, wherein the active feedback comprises a signal sent to a personal electronic device.

Example 9: The device of claim 1, wherein the processing unit is further to determine usage trends.

Example 10: The device of claim 1, wherein the usage data is transmitted to a third party.

Example 11: The device of claim 1, wherein the pressure sensor, network interface and processing units are configured to be in a spacer.

Example 12: The device of claim 1, wherein the pressure sensor, network interface and processing units are configured to be in a mouthpiece.

Example 13: A spacer apparatus comprising: a spacer housing defining a spacer chamber; a pressure sensor to measure flow rate in the spacer; and a processing unit to: calibrate flow rate thresholds for a patient; and provide active feedback to patient during an inhalation based on the calibration.

Example 14: The spacer apparatus of claim 13, wherein the processing unit is further to collect usage data.

Example 15: The spacer apparatus of claim 14, wherein the processing unit is further to transmit usage data.

Example 16: The spacer apparatus of claim 13, wherein the active feedback is auditory, visual, or haptic.

Example 17: An inhaler analysis system comprising: a mouthpiece comprising a pressure sensor to measure flow rate in the spacer; and a spacer comprising a processing unit to: calibrate dosage recommendations for a patient; collect inhalation event data; and transmit, via a network interface, inhalation event data.

Example 18: The inhaler of claim 17, wherein the processing unit is further to provide a dosage reminder.

Example 19: The inhaler of claim 17, wherein to calibrate the dosage recommendations for the patient, the processing unit receives patient information.

Example 20: The inhaler of claim 17, wherein the usage data is transmitted to a physician.

Example 21: An inhalation interface device to collect usage data, the device comprising: a pressure sensor to take measurements including measurements of an air flow rate; a network interface; and a processing unit to: receive a user profile; calibrate the device based on the user profile; collect usage data including the air flow rate; and provide feedback based on the usage data, wherein the feedback is influenced by the calibration.

Example 22: The device of claim 21, wherein the processing unit is further to: update user profile based on the usage data; and re-calibrate based on the updated user profile.

Example 23: The device of claim 21, further comprising additional sensors, and wherein the processing unit is further to measure environmental factors via the additional sensors and calibrate the device based on the environmental factors in addition to the user profile.

Example 24: The device of claim 21, wherein the user profile includes at least one of height, weight, age, prescription data, drug information, and dosage information.

Example 25: The device of claim 21, wherein the processing unit is further to provide a dosage reminder.

Example 26: The device of claim 21, wherein the processing unit is further to provide active feedback to the user during an inhalation, wherein the active feedback is adjusted based on the calibration.

Example 27: The device of claim 26, wherein the active feedback comprises a signal sent to a personal electronic device.

Example 28: The device of claim 21, wherein the processing unit is further to determine usage trends.

Example 29: The device of claim 21, wherein the usage data is transmitted to a third party.

Example 30: The device of claim 21, wherein the pressure sensor, network interface and processing units are configured to be in a spacer of an inhaler.

Example 31: The device of claim 21, wherein the pressure sensor, network interface and processing units are configured to be in a mouthpiece.

Example 32: A inhalation interface apparatus comprising: one or more environmental sensors; a sensor to measure flow rate; and a processing unit to: calibrate the apparatus based on measurements from the environmental sensors; collect usage data including a measurement of flow rate from the pressure sensor; and provide feedback based on the usage data, wherein the feedback is influenced by the calibration.

Example 33: The inhalation interface apparatus of claim 32, wherein the environmental sensors measure one or more of air particulates, location, pollen levels, air quality, atmospheric pressure, atmospheric conditions, temperature, time, and date.

Example 34: The inhalation interface apparatus of claim 32, wherein the feedback comprises active feedback provided during use, wherein the active feedback is auditory, visual, or haptic.

Example 35: The inhalation interface apparatus of claim 32, wherein the processing unit is further to correlate the measurements from the environmental sensors and the measurement of flow rate to generate a user profile that provides a representation of a user during measured environmental conditions, wherein the calibration is further based on the user profile.

Example 36: The inhalation interface apparatus of claim 35, wherein the processing unit calibrates the apparatus by identifying previous environmental conditions recorded in the user profile that are related to the measurements from the environmental sensors, and identifying a flow rate in the user profile that is correlated to the previous environmental conditions.

Example 37: An inhalation analysis system comprising: an inhalation interface comprising a sensor to measure flow rate in the spacer; and a processing unit to: calibrate a usage or dosage recommendation for a user; collect inhalation event data; and transmit, via a network interface, inhalation event data.

Example 38: The inhaler of claim 37, wherein the processing unit is further to provide a usage or dosage reminder.

Example 39: The inhalation analysis system of claim 37, wherein to calibrate the usage recommendations for the patient, the processing unit receives patient information.

Example 40: The inhalation analysis system of claim 37, wherein to calibrate the usage recommendations for the patient, the processing unit receives an inhalable profile.

Example 41: A inhalation interface apparatus comprising: a sensor package including a breath analysis sensor to determine flow rate and biological data of a user; a processing unit to: collect usage data including a measurement of flow rate and biological data; generate a profile of the user based on the usage data, the profile providing a biological status of the user; and provide feedback based on the user profile.

Example 42: The inhalation interface apparatus of claim 42, wherein the sensor package further comprises one or more environmental sensors; and the processing unit is further to: calibrate the apparatus based on measurements from the environmental sensors; and wherein the feedback is influenced by the calibration.

Example 43: The inhalation interface apparatus of claim 42, wherein the environmental sensors measure one or more of air particulates, location, pollen levels, air quality, atmospheric pressure, atmospheric conditions, temperature, time, and date.

Example 44: The inhalation interface apparatus of claim 42, wherein the processing unit is further to correlate the measurements from the environmental sensors and the measurement of flow rate to generate a subset of data in the user profile that provides a representation of a user during measured environmental conditions, wherein the calibration is further based on the user profile.

Example 44: The inhalation interface apparatus of claim 16, wherein the processing unit calibrates the apparatus by identifying previous environmental conditions recorded in the user profile that are related to the measurements from the environmental sensors, and identifying a flow rate in the user profile that is correlated to the previous environmental conditions.

Example 45: The inhalation interface apparatus of claim 41, wherein the feedback comprises active feedback provided during use, wherein the active feedback is auditory, visual, or haptic.

While specific embodiments and applications of the disclosure have been illustrated and described, it is to be understood that the disclosure is not limited to the precise configuration and components disclosed herein. Various modifications, changes, and variations apparent to those of skill in the art may be made in the arrangement, operation, and details of the methods and systems of the disclosure without departing from the scope of the disclosure. The scope of the present disclosure should, therefore, be determined only by the following claims.

1. An inhalation interface device to collect usage data, the device comprising:
a sensor package including a sensor to take measurements including measurements of an air flow rate;
a network interface; and
a processing unit to:
receive a user profile;
calibrate the device based on the user profile;
collect usage data including the air flow rate; and
provide feedback based on the usage data, wherein the feedback is influenced by the calibration.

2. The device of claim 1, wherein the processing unit is further to:
update user profile based on the usage data; and
re-calibrate based on the updated user profile.

3. The device of claim 1, further comprising additional sensors, and wherein the processing unit is further to measure environmental factors via the additional sensors and calibrate the device based on the environmental factors in addition to the user profile.

4. The device of claim 1, wherein the user profile includes at least one of height, weight, age, prescription data, inhalable information, and dosage information.

5. The device of claim 1, wherein the processing unit is further to provide a dosage reminder.

6. The device of claim 1, wherein the processing unit is further to provide active feedback to the user during an inhalation, wherein the active feedback is adjusted based on the calibration.

7. The device of claim 6, wherein the active feedback comprises a signal sent to a personal electronic device.

8. The device of claim 1, wherein the processing unit is further to determine usage trends.

9. The device of claim 1, wherein the sensor package includes one or more sensors that capture biological data of the user, and wherein the processing unit is further to perform an analysis on the biological data to determine a biological profile of the user.

10. The device of claim 1, wherein the usage data is transmitted to a third party.

11. The device of claim 1, wherein the sensor, network interface and processing units are configured to be in a spacer of a respiratory device.

12. The device of claim 1, wherein the sensor, network interface and processing units are configured to be in a mouthpiece.

13. A inhalation interface apparatus comprising:
a sensor package including a breath analysis sensor to determine flow rate and biological data of a user;
a processing unit to:
collect usage data including a measurement of flow rate and biological data;
generate a profile of the user based on the usage data, the profile providing a biological status of the user; and
provide feedback based on the user profile.

14. The inhalation interface apparatus of claim 13, wherein the sensor package further comprises one or more environmental sensors; and
the processing unit is further to:
calibrate the apparatus based on measurements from the environmental sensors; and
wherein the feedback is influenced by the calibration.

15. The inhalation interface apparatus of claim 14, wherein the environmental sensors measure one or more of air particulates, location, pollen levels, air quality, atmospheric pressure, atmospheric conditions, temperature, time, and date.

16. The inhalation interface apparatus of claim 14, wherein the processing unit is further to correlate the measurements from the environmental sensors and the measurement of flow rate to generate a subset of data in the user profile that provides a representation of a user during measured environmental conditions, wherein the calibration is further based on the user profile.

17. The inhalation interface apparatus of claim 16, wherein the processing unit calibrates the apparatus by identifying previous environmental conditions recorded in the user profile that are related to the measurements from the environmental sensors, and identifying a flow rate in the user profile that is correlated to the previous environmental conditions.

18. The inhalation interface apparatus of claim 13, wherein the feedback comprises active feedback provided during use, wherein the active feedback is auditory, visual, or haptic.

19. An inhalation analysis system comprising:
an inhalation interface comprising a sensor to measure inhalation characteristics; and
a processing unit to:
calibrate a usage recommendation for a user;
collect inhalation event data from measurements taken by the sensor; and
transmit, via a network interface, inhalation event data.

20. The inhaler of claim 19, wherein the processing unit is further to provide a usage reminder.

21. The inhalation analysis system of claim 19, wherein to calibrate the usage recommendation for the user, the processing unit receives user information.

22. The inhalation analysis system of claim 19, wherein to calibrate the usage recommendation for the user, the processing unit receives an inhalable profile.
